(19) United States
(12) Patent Application Publication
(10) Pub. No.: US 2008/0009949 A1
(43) Pub. Date: Jan. 10, 2008

(54) Joint Prosthesis and Method of Bone Fixation

(75) Inventors: Rogier Oosterom, The Hague (NL); Albert Johannes Van Der Pijl, The Hague (NL); Harald Erik Niklaus Bersee, Haarlem (NL); Franciscus Cornelus Theod Van Der Helm, Rijswijk (NL); Justus Laurens Herder, Rotterdam (NL)

Correspondence Address: PEACOCK MYERS, P.C., 201 THIRD STREET, N.W., SUITE 1340, ALBUQUERQUE, NM, 87102, US

(73) Assignee: Technische Universiteit Delft, Delft (NL)

(21) Appl. No.: 11/679,409

(22) Filed: Feb. 27, 2007

Related U.S. Application Data
(63) Continuation-in-part of application No. PCT/NL05/00617, filed on Aug. 26, 2005.

(30) Foreign Application Priority Data
Aug. 30, 2004 (NL) 1026925

(51) Int. Cl. A61F 2/30; A61B 17/58

(52) U.S. Cl. 623/18.11; 606/72

(57) ABSTRACT

A joint prosthesis, for example, a knee joint or shoulder joint prosthesis, comprising a first, socket-holding prosthesis part for attachment to a first bone and a second, ball-holding prosthesis part for attachment to a second bone that intermates with the first prosthesis part, wherein the first bone and the second bone are situated at either side of a joint, and wherein the ball of the second prosthesis part is rotatably received in the socket of the first prosthesis part, wherein the first prosthesis part can be coupled with the first bone by applying tensile strain bearing rods, and wherein when the mounted prosthesis is in a virtually unloaded condition there is no significant tensile force in the rods.

### CROSS-REFERENCE TO RELATED APPLICATIONS

This application is a continuation-in-part application of Patent Cooperation Treaty (PCT) Patent Application Serial No. PCT/NL2005/000617, entitled “JOINT PROSTHESIS AND METHOD OF BONE FIXATION” to Technische Universiteit Delft, filed on Aug. 26, 2005, and the specification and claims thereof are incorporated herein by reference.

This Patent Cooperation Treaty (PCT) Patent Application Serial No. PCT/NL2005/000617 application claims priority to and the benefit of the filing of The NETHERLANDS patent application Ser. No. 10/26,925, entitled “JOINT PROSTHESIS AND METHOD OF BONE FIXATION”, filed on Aug. 30, 2004, and the specification and claims thereof are incorporated herein by reference.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### COPYRIGHTED MATERIAL

Not Applicable.

### BACKGROUND OF THE INVENTION

1. Field of the Invention (Technical Field)

The invention relates to a joint prosthesis, for example, a knee joint or shoulder joint prosthesis comprising a first, socket-holding prosthesis part for attachment to a first bone and a second, ball-holding prosthesis part for attachment to a second bone that intermates with the first prosthesis part, wherein the first bone and the second bone are situated at either side of a joint, and wherein the ball of the second prosthesis part is rotatably received in the socket of the first prosthesis part.

2. Description of Related Art

Such a joint prosthesis is known in the form of a shoulder joint prosthesis from the article “Bipolar Shoulder Arthroplasty for painful conditions of the Shoulder”, by Richard L. Worland and Jorge Aradondo, published in The Journal of Arthroplasty, Vol. 13 No. 6 1998, pages 631 to 637.

Both from DE-C-41 33 433 (Schelhas) and EP-A-O 628 294 (Grundel) hip prostheses are known that comprise a socket-like part, and each of which requires the prosthesis design to precisely match the anatomy of the patient.

### BRIEF SUMMARY OF THE INVENTION

Although the joint prosthesis according to the invention is particularly suitable to be applied as shoulder joint prosthesis, the invention is not limited to this application; it is also possible to create other joints with the aid of the joint prosthesis of the invention. Nevertheless, the invention will be elucidated by way of a shoulder joint prosthesis. To the person skilled in the art it is completely obvious how a corresponding operation needs to be performed in, for example, a knee joint or hip joint.

### DETAILED DESCRIPTION OF THE INVENTION

Joint pathology that may result from fractures or joint diseases such as rheumatoid arthritis and arthrosis may seriously diminish the bone quality of the joint surfaces and the stabilizing effect of the surrounding tissue. The joint becomes stiff and painful and will eventually cause chronic pain and will seriously limit the activities of day-to-day life. In order to alleviate the pain and to restore the function of the joint, a total joint reconstruction may be performed. In a conventional joint replacement, both joint surfaces are replaced by a metal ball in the upper arm (humerus) and a polyethylene socket implanted in the shoulder blade (glenoid fossa). Apart from alleviating the pain, the object of this operation is the restoration of the joint stability and the joint functionality. The postoperative results of this procedure depend on, among other things, the quality of the bone and of the surrounding capsule and muscle tissue, on the medical history and age of the patient and on the skill of the surgeon. The most prevalent postoperative complications are instability of the joint and loosening of the glenoid component.

In the case of rheumatoid arthritis there are two important conflicting factors regarding the implantation of a conventional shoulder prosthesis: (1) the lack of the mechanical bone quality necessary for a durable attachment, (2) the loss of the function of surrounding muscles and ligaments of the shoulder, which reduces the necessary joint compression required for stabilizing the joint.

From the above-mentioned prior art the use of a so-called bipolar prosthesis is known. This prosthesis consists of a metal ball that articulates in a conforming synthetic dish. The dish is placed without fixing into a steel shell that is placed against the shoulder blade. The metal ball is placed into the humerus (upper arm). This prosthesis ensures sufficient rotation of the upper arm in relation to the torso, with less wear of the underlying bone. However, the prior art is unable to guarantee stability of the joint.

It is the object of the invention to eliminate this problem and to gain advantages that will be clarified herein-after.

The joint prosthesis according to the invention is characterized in that the first prosthesis part can be coupled with the first bone by applying rods having a tensile strain bearing capacity, wherein when the mounted prosthesis is in a virtually unloaded condition, there is no significant tensile force in the rods.

The rods form artificial ligaments and through translation of the upper arm in any arbitrary direction, they draw the first prosthesis portion against the shoulder blade in such a way as to provide a stabilized power-retaining system.

In a preferred embodiment, the rods divert in the mounted situation preferably in such a manner as to approximately create a virtual point of intersection, which substantially coincides with a stationary point of rotation of the ball of the second prosthesis part in the socket of the first prosthesis part.

As the lines of force in the artificial ligaments formed by the rods coincide with the point of rotation of the second prosthesis part, the forces exerted on the prosthesis do not cause it to rotate.

The stability of the joint prosthesis is enhanced by applying at least three rods having a tensile strain bearing capacity.

It is further beneficial for the stability that the rods having a tensile strain bearing capacity be free of stress of bending.

A further aspect of the joint prosthesis according to the invention concerns the measure that the rods extend through openings in the first prosthesis part and are designed to project through a drilling in the first bone and that at the side of the bone facing away from the first prosthesis part each rod is provided with an enlarged end.

In this way it is possible to fasten the prosthesis in the first bone such as to provide an anchoring of sufficient strength and of a durable nature, also in the case of rheumatoid arthritis.

A first embodiment serving this purpose is characterized in that after insertion of the rods through the drillings of the first bone, said rods are provided with a locking element formed like a sleeve with an inside diameter that is smaller than the diameter of the enlarged end.

A second embodiment is characterized in that each rod is provided with a locking element embodied like a sleeve that is movable around the rods and forms a closed ring, which is provided with anchor arms that in a first position extend along the rods, and being adjustable to a second position, in which said anchor arms have a substantially radial orientation in relation to the rods.

One thing and another makes it possible for the prosthesis according to the invention to be implanted and fastened by means of a one-sided operation. In such a case the operation can then be of a minimally invasive nature.

To serve convenience and simplicity when implanting the prosthesis, each of the rods is provided with thread so that an insert nut can be fitted in a socket of the first prosthesis part.

It is worth noting that fastening the prosthesis by using these rods prevents said artificial ligaments from coming under strain of bending. These rods should only absorb tensile forces. This helps to extend the mechanical life of the prosthesis and avoids post-operative complications.

An above-mentioned embodiment of the locking element to be used for bone fixation is designed like a sleeve that forms a closed ring provided with anchor arms, which in a first position are oriented parallel to the axis of the sleeve body, and being adjustable to a second position in which the anchor arms extend substantially radially from the sleeve.

With this locking element a new method of bone fixation can be applied with which it is also possible to repair bone fractures, and with which it is possible to realize the above-elucidated advantages of tensile strain tolerance in the absence of flexural strain, which would lead to premature loosening of the connecting bone parts.

In this connection, the invention is also embodied in a method of bone fixation, which is characterized in that the bone is provided with at least one drilling, a draw rod provided with an enlarged end is inserted through the drilling, and a locking element is used that prevents the removal of the draw rod from the drilling.

This may be realized, for example, by inserting the locking element into the drilling to prevent the enlarged end from passing through the drilling.

Another possibility is to fit the locking element onto the draw rod, so that the locking element is embodied in accordance with claim 10.

### BRIEF DESCRIPTION OF THE SEVERAL VIEWS OF THE DRAWINGS

Hereinafter the invention will be further elucidated by way of a non-limiting exemplary embodiment and with reference to the drawing.

The drawing shows in:

FIG. 1, an exploded view of the joint prosthesis according to the invention such as can be used as shoulder joint prosthesis;

FIG. 2, the joint prosthesis shown in FIG. 1 in the mounted situation;

FIG. 3, the joint prosthesis according to the invention in an exploded view; and

FIG. 4, the locking element that serves for mounting the joint prosthesis in the pre-mounted and in the mounted situation.

Similar parts in the figures carry identical reference numerals.

### DETAILED DESCRIPTION OF THE INVENTION AND DRAWINGS

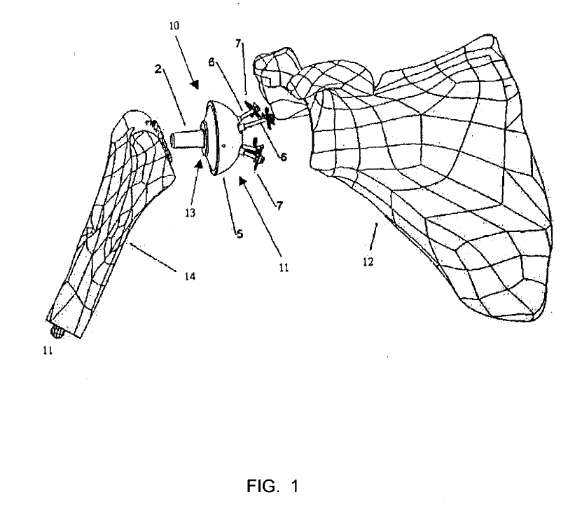

In FIG. 1, the joint prosthesis is indicated with reference numeral 10. This joint prosthesis 10 comprises a first, socket-holding prosthesis part 11 for fastening to a first bone 12, and a second, ball-holding prosthesis part 13 for fastening to a second bone 14, that intermates with the first prosthesis part 11.

In the case shown, the first bone concerns the shoulder blade or the glenoid fossa 12, while the second bone in the case shown is the upper arm or the humerus 14.

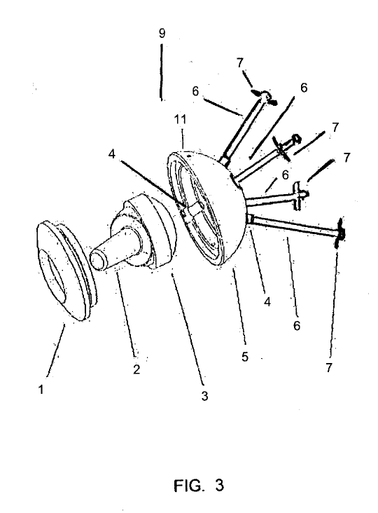

The second prosthesis part 13 comprises a ball 2 that is rotatably received in the socket 5 of the first prosthesis part 11. This can be clearly understood from the exploded view in FIG. 3.

In accordance with the invention, the first prosthesis part 11 can be coupled with the first bone 12 by using three or four rods 6 having a tensile strain bearing capacity.

FIG. 3 clearly shows that in the mounted situation, the at least three rods 6 have a diverging orientation. The divergence of the orientation should be such as to create approximately a virtual point of intersection that substantially coincides with a stationary rotational point of the ball 2 of the second prosthesis part 13 in the socket 5 of the first prosthesis part 11.

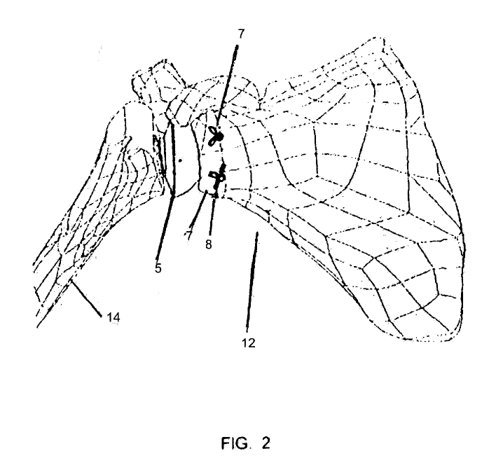

FIG. 2 shows the joint prosthesis in the mounted situation. Viewing FIG. 2 in association with FIG. 3, shows that the at least three rods 6 extend through openings in the socket 5 of the first prosthesis part 11 and are designed to project through a drilling in the first bone 12 and that at the side of the bone 12 facing away from the socket 5, the rods 6 are provided with locking elements 7.

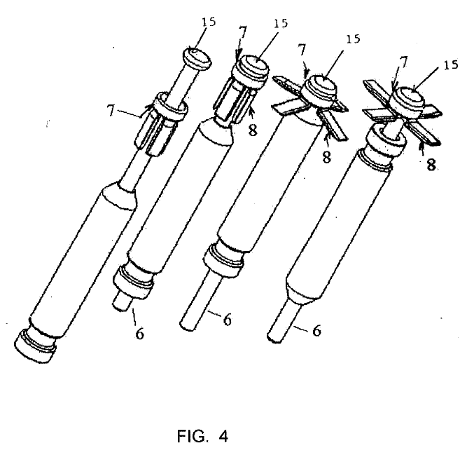

Each of the just-mentioned locking elements 7 is embodied as a sleeve 7, moveable on the artificial ligaments, that is to say the rods 6, and forms a closed ring that in the pre-mounted condition has a first position with anchor arms extending along the rods 6. This is shown by the two sleeves 7 on the left in FIG. 4. The inside diameter of the sleeves 7 is smaller than the diameter of the enlarged end 15 of the rods 6 such that this end 15 forms an obstruction preventing the sleeves 7 from sliding off.

The locking elements 7 can be brought into the mounted situation, as shown in FIG. 2 and like the two sleeves 7 shown on the right in FIG. 4, with the anchor arms 8 extending substantially radially in relation to the rods 6. This is also clearly shown in FIG. 2.

An alternative manner of securing the rods 6 relates to the use of a locking element in the form of a sleeve to be inserted into the bone drillings and having an inside diameter that is smaller than the diameter of the enlarged end 15 of the rods 6. Such sleeves have to be positioned after the insertion of the rods 6. One thing and another is quite obvious to the person skilled in the art so that a further elucidation by way of a figure is not necessary.

FIG. 3 shows that to fasten the rods 6, they can be fixed at the inside of the socket 5 by means of an insert nut 4. To this end the rods 6 are preferably provided with an external thread.

Finally, it is worth noting that in the completed situation, the ball 2 of the second prosthesis part 13 is placed in the socket 5 of the first prosthesis part 11, with the ball 2 preferably being received in a polyethylene inner socket 3. The position of the ball 2 is secured by using a retaining ring 1.

What is claimed is:

1. A joint prosthesis comprising a first, socket-holding prosthesis part for attachment to a first bone and a second, ball-holding prosthesis part for attachment to a second bone that intermates with the first prosthesis part, wherein the first bone and the second bone are situated at either side of a joint, and wherein the ball of the second prosthesis part is rotatably received in the socket of the first prosthesis part, wherein the first prosthesis part can be coupled with the first bone by applying rods having a tensile strain bearing capacity, and wherein when the mounted prosthesis is in a virtually unloaded condition there is no significant tensile force in the rods.

2. A joint prosthesis according to claim 1, wherein there are at least three rods having a tensile strain bearing capacity.

3. A joint prosthesis according to claim 1, wherein the rods having a tensile strain bearing capacity are free of stress of bending.

4. A joint prosthesis according to claim 1, wherein the mounted situation, the rods have a diverging orientation such that they approximately possess a virtual point of intersection that substantially coincides with a stationary rotational point of the ball of the second prosthesis part in the socket of the first prosthesis part.

5. A joint prosthesis according to claim 1, wherein the rods extend through openings in the first prosthesis part and are each designed to project through a drilling in the first bone and that at the side of the bone facing away from the first prosthesis part the rods are provided with an enlarged end.

6. A joint prosthesis according to claim 5, wherein after insertion of the rods through the drillings of the first bone, said rods are provided with a locking element formed like a sleeve with an inside diameter that is smaller than the diameter of the enlarged end.

7. A joint prosthesis according to claim 5, wherein each rod is provided with a locking element embodied like a sleeve that is movable around the rods and forms a closed ring, which is provided with anchor arms that in a first position extend along the rods, being adjustable to a second position, in which said anchor arms have a substantially radial orientation in relation to the rods.

8. A joint prosthesis according to claim 1, wherein each of the rods is provided with thread so that an insert nut can be fitted in a socket of the first prosthesis part.

9. A method of bone fixation, wherein the bone is provided with at least one drilling, a draw rod provided with an enlarged end is inserted through the drilling, and a locking element is used that prevents the removal of the draw rod from the drilling.

10. A method according to claim 9, wherein the locking element is inserted into the drilling to prevent the enlarged end from passing through the drilling.

11. A method according to claim 10, wherein the locking element is provided on the draw rod.
